(19) United States
(12) Patent Application Publication
(10) Pub. No.: US 2026/0248999 A1
(43) Pub. Date: Aug. 27, 2026

(54) INFUSION BAG WITH RINSE PORT, SYSTEMS AND METHODS RELATED TO THE SAME

(71) Applicant: Damea Alexander, Marysville, OH (US)

(72) Inventor: Damea Alexander, Marysville, OH (US)

(21) Appl. No.: 19/065,581

(22) Filed: Feb. 27, 2025

(51) Int. Cl. A61M 5/145; A61M 39/10

(52) U.S. Cl. CPC A61M 5/145; A61M 39/10; A61M 2039/1077

(57) ABSTRACT

An infusion bag for reducing medication waste is disclosed, along with related systems and methods. The infusion bag includes a reservoir for one or more substances for infusing into an individual, a dispensing port is provided at a lower portion of, and is fluidly connected to, the reservoir. A rinse port is provided at an upper portion of, and is fluidly connected to, the reservoir. The reservoir may be filled with the substance(s) for infusion. After infusing all, or substantially all, of the substance(s), a rinse fluid may be introduced through the rise port to pick up remnant substance(s) for infusion, thereby reducing waste.

### CROSS-REFERENCE TO RELATED APPLICATIONS

This application is filed as original and therefore makes no priority claim.

### TECHNICAL FIELD

Exemplary embodiments relate generally to an infusion bag with a rinse port as well as related systems and methods.

### BACKGROUND AND SUMMARY OF THE INVENTION

Infusion is a known medical practice whereby medication or another therapeutic substance is delivered directly into a patient's bloodstream. Bags and other containers (sometimes referred to as “IV bags”), tubing (sometimes referred to as “IV lines”), spikes, vents, drip chambers, connectors (e.g., luer connectors), clamps, needles, intravenous (“IV”) ports, pumps, syringes, and the like are known for facilitating such infusion. Sometime, the infused substances are diluted (e.g., with saline), compounded with other substances, pooled, combinations thereof, or the like, before infusion.

Certain substances, when administered via infusion, may not be completely drained, thereby resulting in waste. For example, some of the substance(s) may be deposited on and/or adhered to inner walls of the reservoir and/or other surfaces of the IV bag (sometimes referred to herein generally as a “bag”), cohered to one another, become trapped on surfaces, combinations thereof, or the like. Some of these substances are relatively expensive and/or otherwise difficult to obtain (e.g., plasma, immunoglobulin, patient specific medication compounds (e.g., compounded chemotherapy medications), short shelf life medication, etc.). Thus, a need exists to ensure that such substances are completely, or more thoroughly, dispensed.

An infusion bag with a rinse port is provided, along with systems and methods related to the same. The rinse port may allow for the introduction of a rinse fluid (e.g., saline) into an internal reservoir of the bag for holding the substance(s). The rinse fluid may be provided to rinse the inner surfaces of the reservoir, thereby collecting some or all of any remnants of the substance(s) therein (e.g., clinging to the inner surfaces by adhesion). The mixed fluid may be subsequently infused into an individual, thereby providing complete, or at least more thorough, substance delivery.

The rinse port is preferably provided at an upper portion of the bag when the bag is oriented for substance administration. The rinse port may comprise a luer lock connector and/or a one-way valve. In exemplary embodiments, the rinse port extends through an upper sealed portion of the bag. A longitudinal axis of the rinse port may be offset from a centerline of the bag and/or reservoir. The longitudinal axis of the rinse port may extend at an angle, such as between about 15 degrees and 75 degrees, from the center of the bag and/or reservoir. The rinse port may optionally comprise features for distributing the rinse fluid, such as ramped surfaces. Any one or more of the foregoing features may allow for complete, or at least more thorough, rinsing. This may, alternatively or additionally, provide ergonomic access and/or a unidirectional, gravity fed flow path for the rinse fluid.

The bag may be filled with one or more substance(s) for infusion, such as by pooling, injection, and/or other known technique(s). The bag may be oriented for infusion. IV lines and/or other equipment may be attached to the bag for infusion, preferably at a dispensing port thereof, which is preferably located at a bottom portion of the reservoir, such as extending through a lower sealed portion of the bag, by way of non-limiting example. The contents of the bag may be infused into the individual, such as by way of the dispensing port and connected IV line and/or other known IV equipment. Preferably once the infusion is complete, or at least substantially complete (e.g., at least 90% emptied, by way of non-limiting example), the bag may be rinsed with the rinse fluid via the rinse port. In this way, some or all of any remnants of the substance(s) (e.g., deposits adhered to the inner walls and/or other surfaces of the reservoir) may be mixed into the rinse fluid, and infused into the individual. The rinse fluid may comprise saline or other pharmaceutically acceptable diluent, by way of example. Rinsing may be performed at other times, multiple times, or the like. The disclosed bag, system, and methods may advantageously be compatible with existing infusion components, systems, and methods, which may be used in whole or in part.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical, similar, or equivalent features, and wherein:

FIG. 1 is front view of an exemplary bag, including an exemplary rinse port;

FIG. 2 is a flow chart with an exemplary method of using the bag of FIG. 1;

FIG. 3 is a plan view of an exemplary system, including the bag of FIG. 1, undergoing the method of FIG. 2; and

FIG. 4 is a detailed, front view of another exemplary embodiment of the rinse port for the bag of FIG. 1, illustrated in isolation.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

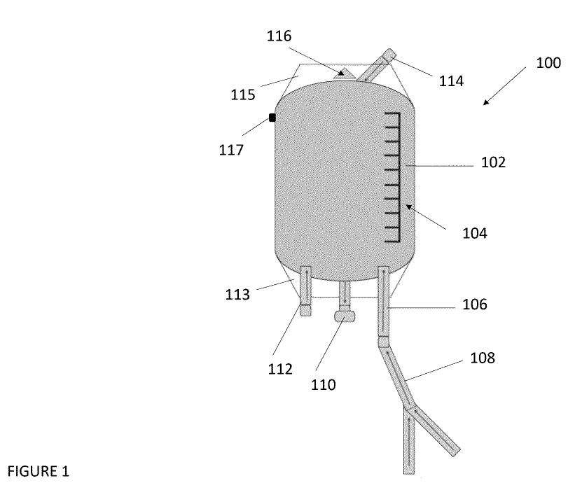

FIG. 1 illustrates an exemplary bag 100. The bag 100 may comprise a reservoir 102. At least the reservoir 102, if not the entire bag 100, may comprise one or more medical grade, flexible materials, such as but not necessarily limited to, ethylene vinyl acetate (EVA), one or more polyolefins (e.g., polycarbonate-based co-polymers), one or more polypropylene-based materials, one or more PVC-free alternatives, combinations thereof, or the like. The reservoir 102 may be configured to hold one or more substances 134 for infusion. The substance(s) 134 may include one or more therapeutics (e.g., medications and/or other active ingredients) and/or one or more inert fluids (e.g., saline and/or other pharmaceutically acceptable diluents and/or other inactive ingredients).

One or more indices 104 may be provided at the reservoir 102, though such is not necessarily required. The indices 104 may comprise, by way of non-limiting example, graduated volumetric markings printed along a longitudinal axis of the bag 100. Other types and kinds of indices 104 may be utilized and arranged in a different manner. Alternatively, the indices 104 may be printed on a separate substrate and affixed to reservoir 102 (e.g., label).

The bag 100 may comprise a pooling port 106, though such is not necessarily required. The pooling port 106 may comprise, by way of non-limiting example, a one way value, luer connector, combinations thereof, or the like.

One or more legs 108 may optionally be provided which form part of, or are removably attachable to, the pooling port 106. The leg(s) 108 may comprise fluid tubing. Preferably, one, two, or three legs is provided, though various number of legs 108 may be utilized. The pooling port 106 and/or leg(s) 108 may be utilized to fluidly connect the reservoir 102 to one or multiple containers, such as for known pooling operations (e.g., relatively complex medication preparations, such as involving drawing a single or multiple substances 134 stored in a single or multiple different containers 126 into the reservoir 102). A distal end of each of the one or more leg(s) 108 may comprise a one way value, luer connector, seal, IV spike connector, a closed, breakable seal, combinations thereof, or the like, such as to maintain sterility before use. The leg(s) 108 may be of various size, shape, and/or type. The leg(s) 108 may be integrally formed with the pooling port 106. Alternatively, the leg(s) 108 may be removably connectable to the pooling port 106, such as by way of a one way value, luer connector, seal, IV spike connector, a closed, breakable seal, combinations thereof, or the like, such as to maintain sterility before use.

The bag 100 may comprise a dispensing port 110. The dispensing port 110 may comprise, by way of non-limiting example, a one way value, luer connector, seal, IV spike connector, a closed, breakable seal, combinations thereof, or the like, such as to maintain sterility and/or fluid storage within the reservoir 102 before use.

The bag 100 may comprise an injection port 112, though such is not necessarily required. The injection port 112 may comprise, by way of non-limiting example, a one way value, luer connector, seal, IV spike connector, a closed, breakable seal, combinations thereof, or the like, such as to maintain sterility and/or fluid storage within the reservoir 102 before use. In exemplary embodiments, without limitation, the injection port 112 may allow a needle to be moved into, through, and/or be otherwise fluidly connected with the injection port 112. In other exemplary embodiments, without limitation, the injection port 112 may allow for a syringe to be fluidly connected to the injunction port 112. In this way, fluids in a syringe (e.g., substance(s) 134) may be injected into the reservoir 102 by way of the injection port 112.

The pooling port 106, dispensing port 110, and/or injection port 112, where such components are utilized, are preferably located at, fluidly connected to, and/or extend generally downward from, a bottom portion of the reservoir 102 when the bag 100 is oriented for infusion. In this way, gravity may assist or cause dispensing of the substance(s) 134 from the reservoir 102, such as through the dispensing port 110 and into connected infusion equipment 124. This may, alternatively or additionally, allow for ergonomic access. The pooling port 106 and/or the injection port 112 may, in particular, be oriented differently, such as at an upper portion of the reservoir 102, side portion of the reservoir 102, or elsewhere.

The bag 100 may comprise a rinse port 114. The rinse port 114 is preferably located at, and extends generally upward from, an upper portion of the reservoir 102 when the bag 100 is oriented for infusion. In this way, gravity may assist or cause the rinse fluid 132 to move through the reservoir 102 to the dispensing port 110. This may allow the rinse fluid 132 to contact some or all of any remnant substance(s) 134, such as deposited along inner walls of the reservoir 102, and/or allow for dispensing of the introduced rinse fluid 132 along with any mixed-in substance(s) 134 remnants through the dispensing port 110. This may, alternatively or additionally, provide ergonomic access to the rinse port 114. The rise port 114 may alternatively be located at, and extend from, other portion of the reservoir 102 (e.g., an upper side portion thereof) when the bag 100 is oriented for infusion.

While a single rinse port 114 is illustrated, multiple rise ports 114 at multiple locations may be utilized, such as to allow different access (e.g., locations, angles, combinations thereof, or the like) for more thorough interior rising.

In exemplary embodiments, without limitation, a longitudinal axis of the rinse port 114 may extend at an angle between about 15 and 75 degrees relative to a longitudinal axis (e.g., centerline) of the bag 100 and/or reservoir 102 thereof, thereby allowing angled ejection of rinse fluid 132 to rinse the reservoir 102 more thoroughly. The rinse port 114 may be off set from the longitudinal axis (e.g., centerline) of the bag 100 and/or reservoir 102, such as to provide ergonomic access. Other locations and/or arrangements may be utilized.

The bag 100 may include a connection component 116, such as an aperture, hook, protrusion, magnet, carabiner, clamp, fastener, combination thereof, or the like, for securing the bag 100 to an external holder 128. The external holder 128 may comprise an aperture, hook, IV stand, protrusion, magnet, carabiner, clamp, fastener, combination thereof, or the like, for connection with the connection component 116. For example, without limitation, the bag 100 may be hung from a hook and/or IV stand by way of the connection component 116, such as by passing the hook (or hook of the IV stand) of the external holder 128 through the aperture of the connection component 116. The connection component 116 is preferably provided at an upper portion of the bag 100 when the bag 100 is oriented for infusion, through multiple connection components 116 may be provided, such as at various locations.

The bag 100 may comprise an upper sealed portion 115 and/or a lower sealed portion 113. The upper and lower sealed portions 115, 113, respectively, may at least partially define the reservoir 102, such as an upper and lower edge thereof. The injection port 112, dispensing port 110, and/or pooling port 106 may extend through the lower sealing portion 113, such as between a lower portion or edge of the reservoir 102 and beyond an outer perimeter of the lower sealing portion 113. The rising port 114 may extend through the upper sealing portion 115, such as between an upper portion or edge of the reservoir 102 and beyond the upper sealing portion 115. In exemplary embodiments, without limitation, the upper sealing portion 115 and the lower sealing portion 113, where provided, may be defined, at least in part, by portions of the bag 100 material which is compressed and sealed to each other, such as by way of heat sealing, by way of non-limiting example. This may advantageously provide a sealed, structurally stable bag 100 and port(s) (e.g., 114, 112, 110, and/or 106). The side edges of the reservoir 102 may be similarly sealed and/or integrally formed by the bag 100. This may provide an efficient form of manufacture.

The reservoir 102 may be defined, at least in part, by a portion of the bag 100 material which is not so sealed, thereby providing the reservoir 102 area, such as for storing the substances 134 and/or other fluids. Advantageously, placing the rinse port 114 at an upper portion of the reservoir 102 and the dispensing port 110 at a lower portion of the reservoir 102 may provide a unidirectional, gravity fed, flow path for fluid drainage. This may be particularly useful for allowing the rinse fluid 132 to pick up any remaining substance(s) 134 to form the mixed fluid for further infusion, thereby reducing waste.

The bag 100 may optionally include one or more air evacuation devices 117. The air evacuation device(s) 117 may comprise a valve (one or two way), vent (one or two way), a removable plug, a removable cap, a removable stopper, a puncturable seal, a luer connection, combinations thereof, or the like. The air evacuation device(s) 117 are preferably provided at an upper portion and/or upper edge of the reservoir 102, such as to allow removal of any air, which may naturally rise within the reservoir 102, such as during dispensing. The air evacuation device(s) 117 may extend through the upper sealing portion 115, side wall of the reservoir 102, combinations thereof, or the like. Vacuums, pumps, syringes, combinations thereof, or the like may be connected to the air evacuation device(s) 117, such as to provide forced air evacuation. Alternatively, or additionally, such air evacuation may be allowed to occur naturally and/or forced manually (e.g., by squeezing the bag 100).

The bag 100, including at least the reservoir 102, indices 104, pooling port 106, leg(s) 108, injection port 110, dispensing port 112, and/or connection component(s) 116, may optionally comprise known and/or conventional reservoir 102, indices 104, pooling port 106, leg(s) 108, injection port 110, dispensing port 112, and/or connection component(s) 116, including but not limited to those available from Right Way Medical, LLC of Westerville Ohio, such as but not limited to, the RWM-10002 product thereof (rightwaymed.com/product/rwm-eva-bag-2-leg-1000 ml/), though such is not required.

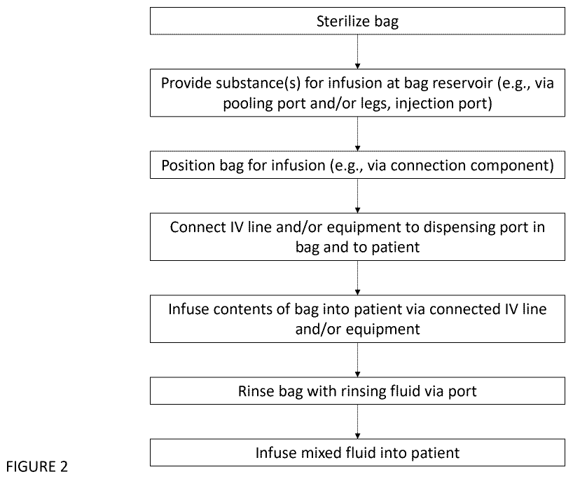

FIG. 2 illustrates an exemplary method of using the bag 100. The bag 100, and/or any infusion equipment 124 to be used therewith, may be sterilized. Sterilization may include ethylene oxide (EtO), gamma irradiation, autoclaving to ensure aseptic integrity, combinations thereof, or the like. One or more substances 134 for infusion, such as medications (including active and/or inactive ingredients thereof), may be provided at the bag 100, specifically into the reservoir 102 thereof. For example, without limitation, some or all of such substance(s) 134 may be provided by way of the pooling port 106 and/or leg(s) 108 using one or more conventional pooling techniques. Alternatively, or additionally, some or all of such substance(s) 134 may be provided by way of the injection port 112, such as using one or more syringes or other connections and injection into the reservoir 102. Alternatively, or additionally, the substance(s) 134 may be provided into the reservoir 102 before sealing the bag 100, such as along the upper and/or lower sealed portions 113, 115 thereof. Known and/or conventional techniques for filling and/or sealing the bag 100 may be utilized, though such is not necessarily required.

The substance(s) 134 may be infused into an individual 122 (e.g., patient) or otherwise dispensed from the reservoir 102 by way of the dispensing port 110 and/or one or more connected infusion equipment items 124, such as various known IV components (e.g., spike, tubing, clamps, drip chambers, needles, IV ports, pumps, monitors, combinations thereof, or the like).

Optionally, such provision of the substance(s) 134 may be provided while the bag 100 is in a first orientation (e.g., laying down, though the bag 100 may move through multiple orientations while the substance(s) 134 are provided), and the bag 100 may be subsequently oriented for infusion (e.g., placed in a second orientation, though the bag 100 may move through multiple orientations infusion is provided), such as by hanging the bag 100 in an upright orientation (e.g., as illustrated with regard to FIG. 1), such as by way of the connection component(s) 116. Such reorientation is preferably performed before infusion, such as before or after connecting the IV lines and/or equipment to the dispensing port 110. Different or various orientations may be used for filling and/or dispensing.

After dispensing some, all, at least a majority, and/or substantially all (e.g., at least 90%) of the substance(s) 134 from the bag 100, the bag 100 may be rinsed. In exemplary embodiments, without limitation, a syringe, luer connector, tubing (e.g., IV line), needle, combinations thereof, or the like is fluidly connected to the rinse port 114. A rinse fluid 132 may be dispensed into the reservoir 102. As used herein, the term rinse fluid 132 include a single or multiple different ingredients. The rinse fluid 132 may comprise one or more biologically inert or substantially biologically inert fluids, such as but not limited to a saline solution, lactated ringer's solution, ringer's solution, dextrose solution, plasma-lyte, buffered salt solutions, water for injection (purified water), colloidal solutions, other pharmaceutically acceptable diluents, combinations thereof, or the like. The rinse fluid 132 is preferably devoid of any medications or other therapeutics, at least before and/or at the time of providing the rinse fluid 132 at the bag 100, to the extent that therapeutics is understood to exclude any biologically inert or substantially biologically inert fluids to the extent that they are understood to provide a therapeutic effect (e.g., saline or other pharmaceutically acceptable diluent that may be used to rehydrate an individual, lower blood pressure, or the like). For example, without limitation, the rinse fluid 132 may be devoid of any active ingredients (e.g., prescribed medications). The rinse fluid 132 may optionally include one or more surfactants considered medically safe for infusion.

The rinse fluid 132 may be provided in various quantities, such as to coat some or all of the inner surfaces of the reservoir 102, preferably reaching at least a majority of such inner surfaces, though any amount of rinsing may be provided. Once provided at the bag 100, the rinse fluid 132 may be mixed with some or all of any remnants of the substance(s) 134. This mixture (of rinse fluid 132 and any picked up remnant substance(s) 134) may be referred to herein as the “mixed fluid”, even if there is no remnants of the substance(s) 134 at the bag 100 and/or no such remnants are picked up by the rinse fluid 132. Some, all, a majority, or substantially all (at least 90%), of the mixed fluid may be infused into the individual 122 by way of the dispensing port 110. Various amounts of rinse fluid 132 may be utilized, such as from a relatively small quantity (e.g., 1% or less of reservoir 102 volume), a relatively large quantity (e.g., to fill the reservoir 102 to its stated capacity or thereabouts (e.g., +/−10% thereof), or some intermediary quantity (e.g., 1-90% of reservoir 102 volume). Rinsing may be performed at various times (e.g., multiple times through the infusion process) and at the same or various quantities. Such rinsing may follow an established protocol for timing and volume, may be provided based on judgment, combinations thereof, or the like.

The steps shown and/or described herein may be provided in various order. For example, without limitation, certain steps may be performed before or after one another, or at the same time. Furthermore, certain steps may be omitted, repeated, or the like.

The foregoing may reduce medication waste while providing customary saline drips for rehydration and/or established vascular access, by way of non-limiting example.

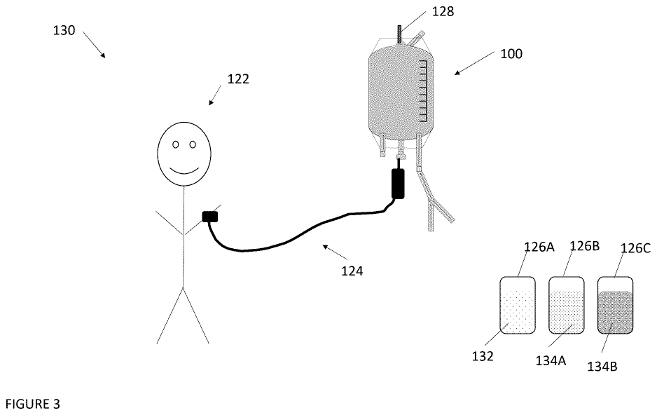

FIG. 3 illustrates the bag 100 in exemplary use as part of an exemplary system 130. One or more infusion equipment items 124 may be provided and connected to the bag 100, such as at the dispensing port 110 thereof. One or more containers 126 (e.g., 126A-126C as illustrated, through various number and type of containers 126 may be utilized), such as containing the substance(s) 134 (e.g., 134A, 134B as illustrated, through various number and type of substances 134 may be utilized, of the same or different type, in same or separate containers 126) and/or the rinse fluid(s) 132 (e.g., various number of rinse fluids 132 may be utilized, of the same or different type, in same or separate containers 126) may be provided. The container(s) 126 may be connected to the bag 100, such as at one or more of the pooling port 106, injection port 112, and/or rinse port 114 thereof. Such connection may be direct or indirect, such as by way of the leg(s) 108 or other infusion equipment 124 (e.g., tubing, spikes, syringes, combinations thereof, or the like). The containers 126 may comprise bottles, bags, combinations thereof, or the like.

The bag 100 may be connected to one or more external holders 128. Preferably, but optionally, the container(s) 126 with the rinse fluid(s) 132 are elevated above the rinse port 114 to allow for natural, gravitational feed, such as by way of an additional one of the external holders 128. However, forced flow (e.g., injection, squeeze, vacuum, or other pressure) may alternatively or additionally be utilized to dispense the rinse fluid(s) 132 into the reservoir 102.

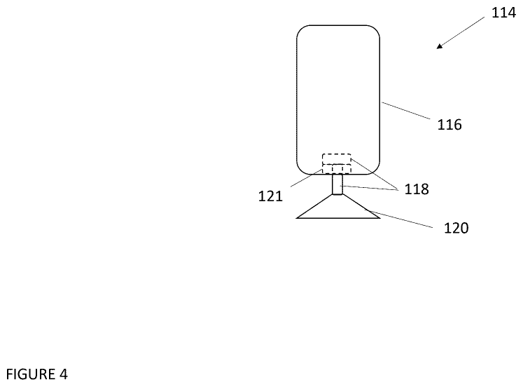

FIG. 4 illustrates a detailed view of an exemplary rinse port 114, by way of non-limiting example. The rinse port 114 may include a body 116, a projection 118, and one or more ramped surfaces 120. The syringe or other component for providing the rinse fluid 132 may be mated with the body 116, such as a luer connection, and the rinse fluid 132 may be provided, preferably under forced flow though natural, gravitational flow may be utilized, through the body 116, thereby contacting the ramped surfaces 120 and causing distribution of the rinse fluid 132 within the reservoir 102. The ramped surfaces 120, the projection 118, and preferably at least part of the body 116 may be located within the reservoir 102 in exemplary embodiments. The ramped surfaces 120, the projection 118, and the body 116 may be integrally formed, or separately formed and connected. Various size, shape, and/or number of projections 118 and/or ramped surfaces 120 may be utilized. The relative position of the ramped surfaces 120 may be fixed. Alternatively, or additionally, the projection 118 and/or ramped surfaces 120 may be biased inward and may be movable, such as by way of a spring 121 located interior to the body 116 and preferably extending about at least part of the projection 118 and contacting a lower surface of the body 116 and an upper portion of the projection 118.

Notably, these embodiments (i.e., shown and/or described with regard to FIG. 4) are optional and not intended to be limiting.

Other embodiments of the rinse port 114 may include, by way of non-limiting example, luer connections, needle ports, seals (which may be broken by a needle or the like), combinations thereof, or the like. This may provide for sterility of the bag 100 and optional use of the rise port 114 by users (e.g., healthcare providers).

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention.

What is claimed is:

1. An infusion bag for reducing medication waste, said infusion bag comprising:
a reservoir for one or more substances for infusing into an individual;
a dispensing port provided at a lower portion of, and fluidly connected to, said reservoir; and
a rinse port provided at an upper portion of, and fluidly connected to, said reservoir.

2. The infusion bag of claim 1 wherein:
the rinse port comprises a luer connector.

3. The infusion bag of claim 1 wherein:
the rinse port comprises a one way valve.

4. The infusion bag of claim 1 wherein:
the rinse port comprises a body, a projection extending from said body, and a ramped surface connected to a distal end of said projection; and
said ramped surface is located within said reservoir.

5. The infusion bag of claim 1 further comprising:
an injection port provided at the lower portion of, and fluidly connected to, said reservoir.

6. The infusion bag of claim 1 further comprising:
a pooling port provided at the lower portion of, and fluidly connected to, said reservoir.

7. The infusion bag of claim 6 further comprising:
legs connected to, and extending from, the pooling port.

8. The infusion bag of claim 1 further comprising:
an upper sealing portion defining, at least in part, an upper edge of the reservoir, wherein the rising port extends through the upper sealing portion; and
a lower sealing portion defining, at least in part, a lower edge of the reservoir, wherein the dispensing port extends through the lower sealing portion.

9. The infusion bag of claim 8 further comprising:
a connection component provided at the upper sealing portion of the bag, said connection component comprising a through aperture.

10. The infusion bag of claim 1 further comprising:
indices provided along a longitudinal axis of the reservoir.

11. The infusion bag of claim 1 wherein:
a longitudinal axis of the rinse port is offset from a longitudinal axis of the bag.

12. The infusion bag of claim 1 wherein:
a longitudinal axis of the rinse port extends at an angle between 15 degrees and degrees from a longitudinal axis of the bag.

13. The infusion bag of claim 1 further comprising:
an air evacuation device fluidly connected to an upper portion of the reservoir.

14. A system for reducing medication waste, said system comprising:
the infusion bag of claim 1; and
one or more items of infusion equipment.

15. A method for reducing medication waste, said method comprising:
providing the infusion bag of claim 1;
providing the one or more substances within the reservoir for infusion into an individual, said one or more substances each comprising at least one active therapeutic ingredient;
after and/or while infusing all, or substantially all, of the one or more substances by way of the dispensing port, introducing a rinse fluid into the reservoir by way the rinse port, said rinse fluid comprising saline or another pharmaceutically acceptable diluent; and
infusing a mixed fluid comprising the rinse fluid and remnants of the one or more substances from the reservoir into the individual by way of the dispensing port.

16. The method of claim 15 further comprising:
sterilizing the bag;
connecting one or more items of infusion equipment between the dispensing port and the individual; and
positioning the bag for infusion.

17. The method of claim 15 wherein:
the step of introducing the rinse fluid into the reservoir by way the rinse port comprises coating all or substantially all of the inner surfaces of the reservoir with the rise fluid; and
said rinse fluid is devoid of any prescribed medications.

18. The method of claim 15 wherein:
providing the one or more substances within the reservoir for infusion into an individual comprises one or more of:
pooling at least one of the one or more substances into the reservoir by way of a pooling port provided at the lower portion of, and fluidly connected to, said reservoir; and
injecting at least one of the one or more substance into the reservoir by way of an injection port provided at the lower portion of, and fluidly connected to, said reservoir.

19. An infusion bag for reducing medication waste, said infusion bag comprising:
a reservoir;
an upper sealing portion defining, at least in part, an upper edge of the reservoir;
a lower sealing portion defining, at least in part, a lower edge of the reservoir;
a dispensing port provided at a lower portion of, and fluidly connected to, said reservoir, wherein said dispensing port extends through the lower sealing portion;
a rinse port provided at an upper portion of, and fluidly connected to, said reservoir, wherein said rinse port comprises a luer connector or a one way valve;
an injection port provided at the lower portion of, and fluidly connected to, said reservoir; and
a pooling port provided at the lower portion of, and fluidly connected to, said reservoir.

20. The infusion bag of claim 19:
wherein a longitudinal axis of the rinse port is offset from a longitudinal axis of the bag;
wherein the longitudinal axis of the rinse port and extends at an angle between 15 degrees and 75 degrees from the longitudinal axis of the bag;
further comprising a connection component comprising a through aperture provided at the upper sealing portion of the bag;
further comprising legs connected to, and extending from, the pooling port; and
further comprising indices provided along a longitudinal axis of the reservoir.
